(19) United States
(12) Patent Application Publication
(10) Pub. No.: US 2020/0407301 A1
(43) Pub. Date: Dec. 31, 2020

(54) FRAGRANCE AND FLAVOR MATERIALS DERIVED FROM 1-(4-METHYLCYCLOHEX-1-EN-1-YL)ETHAN-1-ONE

(71) Applicant: TAKASAGO INTERNATIONAL CORPORATION, Tokyo (JP)

(72) Inventors: Maureen Blandino, Dumont, NJ (US); Michael E. Lankin, High Bridge, NJ (US); Louis J. Lombardo, Washingtonville, NY (US)

(73) Assignee: TAKASAGO INTERNATIONAL CORPORATION, Tokyo (JP)

(21) Appl. No.: 16/978,988

(22) PCT Filed: Mar. 8, 2019
(86) PCT No.: PCT/US2019/021457

§ 371 Date: Sep. 8, 2020

Related U.S. Application Data
(60) Provisional application No. 62/640,499, filed on Mar. 8, 2018.

(51) Int. Cl. C07C 43/162; C07C 251/18; C07D 317/12; C07C 69/007; C07C 69/96

(52) U.S. Cl. CPC C07C 43/162; C07C 251/18; C11D 7/261; C07C 69/007; C07C 69/96; C07D 317/12

(57) ABSTRACT

The present disclosure is directed to 1-(4-methyl cyclohex-1-en-1-yl)ethan-1-one derivatives having unique and desired flavor and/or fragrance characteristics, as well as the synthesis and application of the derivatives. The compounds of the present disclosure can be employed alone or incorporated as fragrance or flavor ingredients to modify or enhance already existing fragrance compositions, solvents, media, and the like.

### CROSS-REFERENCE TO RELATED APPLICATIONS

This application claims priority to U.S. Provisional Patent Application Ser. No. 62/640,499, filed Mar. 8, 2018, the contents of which are incorporated by reference herein in its entirety.

### FIELD OF THE INVENTION

The present application relates to compounds useful as fragrance or flavor components in fragrance or flavor compositions.

### BACKGROUND OF THE INVENTION

There is a continuing interest in the preparation new of synthetic fragrance and flavor components with unique organoleptic properties and their use in consumer products. Differences in chemical structures can significantly impact odor, notes, and other organoleptic, chemical, and physical characteristics. Thus, there is a continuous need for new chemical structures that have favorable organoleptic properties. Once identified, these novel chemical compounds can provide perfumers and other persons with the ability to create new, unique fragrances.

There is also a need for more efficient synthesis techniques to prepare components with pleasing and consumer preferred fragrance and flavor compositions. One strategy to prepare such compounds is to make derivatives of a chemical entity. 1-(2-hydroxy-4-methylcyclohexyl)-ethanone is such a chemical entity.

The presently disclosed subject matter addresses these and other needs by providing new fragrance compounds with unique and desirable organoleptic properties, as discussed in detail below. Additionally, synthetic procedures for making such compounds are disclosed herein.

### SUMMARY OF THE INVENTION

The present disclosure is directed to derivatives of 1-(4-methyl cyclohex-1-en-1-yl)ethan-1-one having unique and desired organoleptic characteristics, and synthesis and uses thereof. The compounds of the present disclosure can be employed alone or in combination with additional compounds. The compounds of the present disclosure can be incorporated into fragrance or flavor compositions.

In certain embodiments, the presently disclosed subject matter provides the compound represented by Formula I:

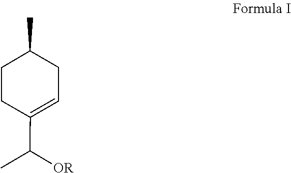

where R is H, C₁-C₈ alkyl, C₁-C₈ alkenyl, C(O)R₁, or C(O)OR₁, and

where R₁ is selected from the group consisting of H, C₁-C₈ alkyl, C₁-C₈ branched alkyl, C₁-C₈ alkenyl, C₁-C₈ branched alkenyl, C₁-C₈ cycloalkyl, aryl, and a substituted aryl.

In another embodiment, the presently disclosed subject matter provides the compound represented by Formula II:

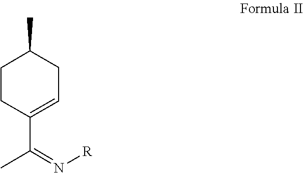

where R is H or OR₁, and
where R₁ is selected from the group consisting of C₁-C₈ alkyl, C₁-C₈ branched alkyl, C₁-C₈ alkenyl, C₁-C₈ branched alkenyl, and C(O)R₂; and R₂ is selected from the group consisting of H, C₁-C₅ alkyl, C₁-C₅ branched alkyl, C₁-C₅ alkenyl, and C₁-C₅ branched alkenyl.

In another embodiment, the present disclosure also provides a compound represented by Formula III:

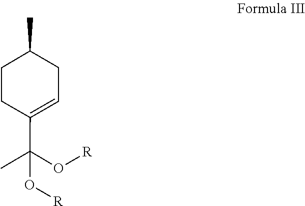

where each R is independently H, C₁-C₈ alkyl, C₁-C₈ branched alkyl, C₁-C₈ alkenyl, or C₁-C₈ branched alkenyl.

In another embodiment, the present disclosure also provides a compound represented by Formula IIIa:

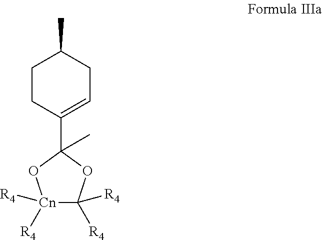

wherein n is 1 or 2;

wherein each R₄ is independently H, C₁-C₈ alkyl, C₁-C₈ branched alkyl, C₁-C₈ alkenyl, or C₁-C₈ branched alkenyl.

The presently disclosed subject matter also provides for fragrance compositions comprising at least one compound disclosed herein. In a particular embodiment of a fragrance composition, the concentration of the at least one compound is from about 0.001% to about 20% by weight of the fragrance composition.

In certain embodiments, the fragrance composition further comprises one or more compounds selected from the group consisting of one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

The presently disclosed subject matter also provides for flavor compositions comprising at least one compound disclosed herein. In a particular embodiment of a flavor composition, the concentration of the at least one compound is from about 0.0001% to about 20% by weight of the flavor composition. In certain embodiments, the flavor composition further comprises a flavor carrier.

In certain embodiments, the compounds include constitutional isomers, enantiomers, stereoisomers, and racemic mixtures of said compounds listed herein.

Another aspect of the present disclosure provides a fragrance or flavor composition for addition to a consumer product comprising one or more one the presently disclosed compounds in an amount effective to impart a fragrance or flavor to the consumer product.

### DETAILED DESCRIPTION

As noted above, there remains a need and demand in the art for unique fragrance and flavor compositions. There is also a need for more efficient synthesis techniques to prepare components with pleasing and consumer preferred fragrance and flavor compositions. The presently disclosed subject matter addresses these needs through compounds disclosed herein and/or new fragrance and flavor compositions comprising one or more of the disclosed compounds.

For clarity, and not by way of limitations, the detailed description is divided into the following subsections:

1. Definitions;

2. Derivatives of 1-(4-methyl cyclohex-1-en-1-yl)ethan-1-one;

3. Synthesis of Derivatives of 1-(4-methyl cyclohex-1-en-1-yl)ethan-1-one;

4. Fragrance Compositions;

5. Use of Fragrance Compositions in Consumer Products;

6. Flavor Compositions;

7. Use of Flavor Compositions in Consumer Products.

### 1. Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “enantiomers” refers to a pair of stereoisomers that are non-superimposable mirror images of each other. A 1:1 mixture of a pair of enantiomers is a “racemic” mixture. The term is used to designate a racemic mixture where appropriate.

As used herein, the term “diastereoisomers” refers to stereoisomers that have at least two asymmetric atoms, but which are not mirror-images of each other. The absolute stereochemistry is specified according to the Cahn-Ingold-Prelog R S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and can thus give rise to enantiomers, diastereomers, and other stereoisomeric forms that can be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers can be prepared using chiral synthons or chiral reagents, or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

As used herein, the term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Therefore, the presently disclosed subject matter includes enantiomers, diastereomers, or racemates of the compound. Also, as used herein, the terms “constitutional isomers” refers to different compounds which have the same numbers of, and types of, atoms but the atoms are connected differently.

As used herein, the term “fragrance composition” refers to a mixture comprising one or more fragrance components, in any of their forms, and one or more solvents or perfuming co-ingredients. As known in the art, a fragrance composition contains one or more fragrance components (e.g., perfuming co-ingredients) in order to impart an olfactory note to the composition (e.g., a household cleaner, perfume, or other consumer product) to which it is added. In one embodiment, the fragrance composition contains two or more fragrance components which, collectively and in combination with the solvent to which they are added, impart an intended olfactory note (e.g., a hedonically pleasing “tropical” note) to a human in close proximity to the fragrance composition.

In general terms, perfuming co-ingredients belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin, and are known to perfumists of ordinary skill in the art. Many of these ingredients are listed in reference texts such as S. Arctander, Perfume and Flavor Chemicals, 1969, Montclair, N.J., USA or any of its more recent versions, each of which are hereby incorporated by reference.

As used herein, the term “flavor composition” refers to a composition that contains one or more compounds (e.g., co-ingredients) that provide a desired taste when combined with a solvent that is suitable for oral administration and oral consumption. Examples of flavoring co-ingredients that are generally included in a flavor composition are listed in S. Arctander, Perfume and Flavor Chemicals, 1969, Montclair, N.J., USA. The skilled person in the art of flavors is able to select them on the basis of its general knowledge and according to the nature of the product to be flavored and the desired taste.

As used herein, the phrase “consumer product” or “end product” refers to composition that is in a form ready for use by the consumer for the marketed indication. A solvent suitable for use in a consumer product is a solvent that, when combined with other components of the end product, will not render the consumer product unfit for its intended consumer use.

### 2. Derivatives of 1-(4-methyl cyclohex-1-en-1-yl)ethan-1-one

The compound, 1-(4-methyl cyclohex-1-en-1-yl)ethan-1-one, is a by-product of the synthesis of 3,6-dimethyl-5,6,7,7a-tetrahydrobenzofuran-2(4H)-one according to the method described in PCT Patent Application Publication No. WO2017/044957,

incorporated herein in its entirety by reference. Alternatively, 1-(4-methyl cyclohex-1-en-1-yl)ethan-1-one can be prepared directly in a single step from 1-(2-hydroxy-4-methylcyclohexyl)-ethanone by dehydration according to the method described in PCT Patent Application Publication No. WO2017/044957. 1-(4-methyl cyclohex-1-en-1-yl)ethan-1-one can also be prepared according to a method described by Sadygov in “Study of induced hypohalogenation of 1-alkyl(4-methylcyclohex-1-enyl)alkanones, and chemical transformations of the obtained compounds” published in Russian Journal of General Chemistry, 79(8), 1691-1697 (2009), and incorporated herein in its entirety by reference.

Starting from 1-(4-methyl cyclohex-1-en-1-yl)ethan-1-one, novel fragrance and flavor compounds can be derived, i.e., alcohols, ethers, esters, ketones, ketals, and oximes of 1-(4-methyl cyclohex-1-en-1-yl)ethan-1-one. In certain embodiments, the presently disclosed subject matter provides derivative compounds that are alcohols. In certain embodiments, the presently disclosed subject matter provides derivative compounds that are ethers. In certain embodiments, the presently disclosed subject matter provides derivative compounds that are esters. In certain embodiments, the presently disclosed subject matter provides derivative compounds that are ketals. In certain embodiments, the presently disclosed subject matter provides derivative compounds that are oximes. Nonlimiting representative examples of these compounds are shown in the following Formulas.

In certain embodiments, the presently disclosed subject matter provides the compound of Formula I below:

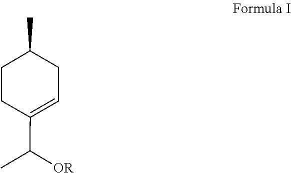

where R is H, C₁-C₈ alkyl, C₁-C₈ alkenyl, C(O)R₁, or C(O)OR₁; and where R₁ is selected from the group consisting of H, C₁-C₈ alkyl, C₁-C₈ branched alkyl, C₁-C₈ alkenyl, C₁-C₈ branched alkenyl, C₁-C₈ cycloalkyl, aryl, and a substituted aryl.

In another embodiment, the presently disclosed subject matter provides the compound of Formula II below:

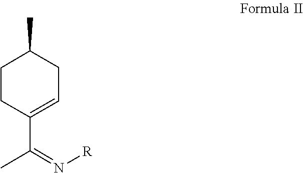

where R is H or OR₁, and
where R₁ is selected from the group consisting of C₁-C₈ alkyl, C₁-C₈ branched alkyl, C₁-C₈ alkenyl, C₁-C₈ branched alkenyl, and C(O)R₂; and R₂ is selected from the group consisting of H, C₁-C₅ alkyl, C₁-C₅ branched alkyl, C₁-C₅ alkenyl, and C₁-C₅ branched alkenyl.

In certain embodiments, the presently disclosed subject matter provides the compound of Formula III below:

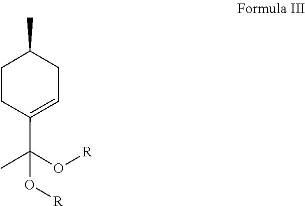

where each R is independently H, C₁-C₈ alkyl, C₁-C₈ branched alkyl, C₁-C₈ alkenyl, or C₁-C₈ branched alkenyl.

In an alternative embodiment, the presently disclosed subject matter provides a compound represented by Formula IIIa:

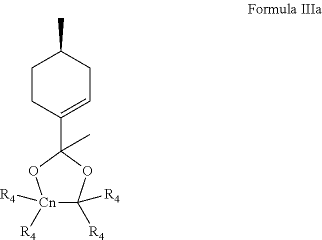

wherein n is 1 or 2;

wherein each R₄ is independently H, C₁-C₈ alkyl, C₁-C₈ branched alkyl, C₁-C₈ alkenyl, or C₁-C₈ branched alkenyl.

In a particular embodiment of the present disclosure, the compound can be 1-((R)-4-methylcyclohex-1-en-1-yl)ethan-1-ol.

In particular embodiment of the present disclosure, the compound can be 1-((R)-4-methylcyclohex-1-en-1-yl)ethyl propionate.

In another particular embodiment of the present disclosure, the compound can be 1-((R)-4-methylcyclohex-1-en-1-yl)ethyl cyclopropanecarboxylate acetate.

In particular embodiments of the present disclosure, the compound can be 1-((R)-4-methylcyclohex-1-en-1-yl)ethylacetate, 1-((R)-4-methylcyclohex-1-en-1-yl)ethyl benzoate, (4R)-1-(1-methoxyethyl)-4-methylcyclohex-1-ene, (R)-1-(4-methylcyclohex-1-en-1-yl)ethan-1-one oxime, or (R)-2-methyl-2-(4-methylcyclohex-1-en-1-yl)-1,3-dioxolane.

### 3. Synthesis of derivatives of 1-(4-methyl cyclohex-1-en-1-yl)ethan-1-one

The presently disclosed compounds can be prepared synthetically through various chemical transformations. The Examples provided here describe various nonlimiting embodiments of synthetic procedures. The compounds that are alcohols can be prepared using a reducing agent such as sodium borohydride or lithium aluminum hydride. The compounds that are ethers can be prepared using an alkyl halide, such as methyl iodide, in the presence of a strong base, such as sodium hydride. The compounds that are esters can be prepared using an anhydride, such as acetic anhydride, or acyl chlorides, or organic acids. The compounds that are acetals and ketals can be prepared using a diol, such as ethylene glycol or propylene glycol, or other substituted 1,2-diols in the presence of a weak organic acid such as propionic acid or p-toluenesulfonic acid. The compounds that are oximes can be prepared using a hydroxy amine salt, such as hydroxylamine hydrochloride.

### 4. Fragrance Compositions

In certain embodiments, any one of the above described compounds can be provided in a fragrance composition. Certain embodiments of the presently disclosed subject matter provide a method to modify, enhance or improve the odor properties of a fragrance composition by adding to the composition an effective quantity of one or more of the compounds.

For fragrance applications, concentrations of the compounds of Formulas (I-III) are based on the total weight of the composition into which the fragrance compound is incorporated. For fragrance applications, typical concentrations of the presently disclosed compounds range from about 0.001% to about 20% by weight, or from about 0.01% to about 10% by weight, or from about 0.1% to about 5%, or from about 1% to about 3%, based on the total weight of the composition into which the fragrance compound is incorporated. Those skilled in the art are able to employ the desired level of the compounds of the disclosed subject matter to provide the desired fragrance/flavor and intensity.

The compounds of the presently disclosed subject matter can be combined with one or more fragrance accords or compounds from various fragrance categories including but not limited to one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

Non-limiting examples of suitable aldehydic compounds include acetaldehyde C-8, acetaldehyde C-9, acetaldehyde C-10, adoxal, aldehyde C-8, aldehyde C-9, aldehyde C-10, aldehyde C-11, aldehyde C-12, aldehyde C-12 lauric, aldehyde C-12 MNA, aldehyde supra, cyclomyral trans-2-decenal, trans-4-decenal, cis-4-decenal, 9-decenal, myrac aldehyde, precyclemone B, trans-2-dodecenal, undecylenic aldehyde, 1-methyl-4-(4-methylpentyl)cyclohex-3-ene-1-carbaldehyde (VERNALDEHYDE®), and combinations thereof.

Non-limiting examples of an animalic compound are 5-Cyclohexadecen-1-one (AMBRETONE®), 17-oxacycloheptadec-6-en-1-one (ambrettolide), 2,5,5-trimethyl-1,3,4,4a,6,7-hexahydronaphthalen-2-ol (ambrinol), 2-Methyl-5-(5,5,6-trimethylbicyclo[2.2.1]hept-2-yl)cyclohexanone (ALDRON®), civet, p-cresol, cresyl methyl ether, indole, skatole, and combinations thereof.

Non-limiting examples of a balsamic compound are benzy salicylate, cylohexyl salicylate, isopropoxy ethyl salicylate, phenethyl salicylate, styrax oil, and combinations thereof.

Non-limiting examples of a citrus compound are delta-3-carene, citral, citronellal, L-cintronellol, decanal, DH-L-citronellol, myrcenol, limonene, DH-myrcenol, nootkatone, sinensal, rhubafuran, bergamot oil, grapefruit oil, lemon oil, lime oil, orange oil, mandarin oil tridecene-2-nitrile, and/or yuzu core base.

Non-limiting examples of a floral compound are acetanisole, alpha amyl cinnamaldehyde, anisyl acetate, anisic aldehyde, benzyl acetate, bourgeonal, butyl acetate, hexyl cinammic aldehyde, 1-citrol, cyclamen aldehyde, cyclohexyl lactone, delta-damascone, 9-decen-1-ol, dimethyl benzyl carbinol, farnesal, 1-dihydrofarnesal, ethyl linalool, 1-farnesal, farnesol, 1-dihydrofarnesol, 3-(3-Isopropylphenyl)butanal (FLORHYDRAL®), 3-(4-ethylphenyl)-2,2-dimethylpropanal (floralozone), 4-methyl-2-(2-methylpropyl)oxan-4-ol (FLOROL®), geraniol, gernayl acetate, piperonal, methyl 3-oxo-2-pentylcyclopentaneacetate (Hedione®), 2-Methyl-3-(3,4-methylenedioxyphenyl)propanal (Heliobouquet), hexyl cinnamaldehyde, hexyl salicylate, indole, alpha-ionone, beta-ionone, isopropoxy ethyl salicylate, methyl-2-((1S*,2R*)-3-oxo-2-pentylcyclopentyl)acetate (JASMODIONE®), cis-jasmone, 4-(4-hydroxy-4-methylpentyl)cyclohex-3-ene-1-carbaldehyde (KOVANOL®), laurinal, lilial, linalool, linalyl acetate, 2,4,6,-trimethyl-4-phenyl-1,3-dioxane (LOREXAN®), 2,4-Dimethyl-4,4a,5,9b-tetrahydroindeno[1,2-d][1,3]dioxine (Magnolan), (4-propan-2-ylcyclohexyl)methanol (Mayol), methyl dihydrojasomante, gamma-methyl ionone, methoxymelonal, methyl benzoate, 1-(4-Isopropyl-cyclohexyl) ethanol (Mugetanol), nerol, 1-(3-Methyl-benzofuran-2-yl)-ethanone (Nerolione), neryl acetate, orantha, L., 2-pentyl cyclopentanone, 2-cyclohexyl-2-cyclohexylideneacetonitrile (PEONILE®), phenoxanol, phenoxy ethyl isobutyrate, phenylacetaldehyde, phenyl ethyl alcohol, prenyl salicylate, rose oxide, rosephenone, rosyrane, suzaral, terpineol, undecavertol, 2,2,5-trimethyl-5-pentylcyclopentan-1-one (VELOUTONE®), yara yara, geranium oil, rose oil, lavender oil, ylang oil, and combinations thereof.

Non-limiting examples of a fruity compound are aldehyde C-16, allyl caproate, allyl cyclohexyl proprionate, allyl heptanoate, amyl acetate, benzaldehyde, cassis oil, L-citronellyl acetate, L-citronellyl nitrile, 3a,4,5,6,7,7a-hexahydro-4,7-methano-1h-inden-5(or 6)-yl acetate (CYCLACET®), 3a,4,5,6,7,7a-hexahydro-1H-4,7-methanoinden-1-yl propanoate (CYCLAPROP®), damascenone, beta-decalactone, gamma-decalactone, diethyl malonate, dimethyl benzyl carbinol acetate, dimethyl benzyl carbinyl butyrate, dimethyl phenyl ethyl carbinol, dimethyl sulfide, γ-dodecalactone, ethyl acetate, ethyl butyrate, ethyl caproate, ethyl decadienotate, ethyl heptoate, ethyl-2-methylbutyrate, ethyl acetoacetate, ethyl methyl phenyl glycidate, ethyl propionate, 4-methyl-2-(2-methylpropyl)oxan-4-ol (FLOROL®), ethyl tricyclo [5.2.1.02.6] decan-2 carboxylate (FRUITATE®), hexyl acetate, hexyl isobutyrate, isoamyl acetate, 6-(pent-3-en-1-yl)tetrahydro-2H-pyran-2-one (Jasmolactone), ethyl 2-methylpentanoate (manzanate), 2,6-dimethylhept-5-enal (melonal), methyl anthranilate, methyl dioxolan, methyl heptyl ketone, gamma-nonalactone, 6-nonenol, gamma-octalactone, phenyl ethyl isobutyrate, prenyl acetate, raspberry ketone, methyl(2-((4S)-4-methyl-2-methylenecyclohexyl)propan-2-yl)sulfane (RINGONOL®), (1R,6S)-ethyl 2,2,6-trimethylclocloxehanecarboxylate (THESARON®), tolyl aldehyde, γ-undecalactone, 3,5,5-trimethylhexyl acetate (vanoris), (2-tert-butylcyclohexyl) acetate (verdox), and combinations thereof.

Non-limiting examples of a gourmand compound are angelica lactone-alpha, caprylic acid, coumarin, ethyl fraison, ethyl vanillin, ethyl maltol (e.g., VELTOL PLUS), filbertone, 4-hydroxy-2,5-dimethyl-3(2H)-furanone (FURANEOL®), guaiacol, maple furanone, 2-acetyl pyrazine, 2,5-dimethyl pyrazine, vanillin and combinations thereof.

Non-limiting examples of a green compound are allyl amyl glycolate, cyclogalbanate, 1-(5,5-dimethyl-1-cyclohexen-1-yl)-4-Penten-1-one (DYNASCONE®), galbanolene, galbanum, trans-2-hexenal, cis-3-hexenol, hexen-1-ol, cis-3-hexenyl acetate, cis-3-hexenyl butyrate, cis-3-hexenyl formate, cis-3-hexenyl salicyclate, liffarome, 2-methoxy-2-methylheptane, methyl octine carbonate, neofolione, 2,6-nonadienal, (2R,4S)-2-methyl-4-propyl-1,3-oxathiane (OXANE®), octahydro-5-methoxy-4,7-methano-1H-indene-2-carboxaldehyde (SCENTENAL®), N-(5-methylheptan-3-ylidene)hydroxylamine (STEMONE®), styrallyl acetate, 2,4-dimethylcyclohex-3-ene-1-carbaldehyde (TRIPLAL®), undecavertol, vionil, violet methyl carbonate (e.g., VIOLET T), violet leaf extract, and combinations thereof.

Non-limiting examples of an herbaceous compound are anethol, bamboo ketone, canthoxal, carvacrol, carvone, 1, clary sage natural oil, cymene, p., Daikon Ether, 2,6-dimethylheptan-2-ol (DIMETOL®), menthol, methyl salicylate, thymol, natural basil oil, natural eucalyptus oil, eucalyptol, sweet natural fennel oil, natural cedar leaf oil, and combinations thereof.

Non-limiting examples of a marine compound are 8-methyl-1,5-benzodioxepin-3-one (Calone® 1951), 3-(4-ethylphenyl)-2,2-dimethylpropanal (floralozone), 4-tert-butylphenylacetonitrile (MARENIL®), 4-[(3E)-4,8-dimethylnona-3,7-dienyl]pyridine (MARITIMA), myrac aldehyde, ultrazure, and combinations thereof.

Non-limiting examples of a mossy compound are hinokitiol, isobutyl quinolone, isopropyl quinolone and/or methyl 2,4-dihydroxy-3,6-dimethylbenzoate (Oakmoss™ #1), and combinations thereof.

Non-limiting examples of a musk compound are 17-oxacycloheptadec-6-en-1-one (ambrettolide), 5-Cyclohexadecen-1-one (AMBRETONE®), (3aR,5aS,9aS,9bR)-3a,6,6,9a-Tetramethyldodecahydronaphtho[2,1-b]furan (AMBROXAN), 2,2,6-trimethyl-alpha-propylcyclohexanepropanol (Dextramber), 16-oxacyclohexadecan-1-one (EXALTOLIDE®), galaxolide, ((12E)-1-oxacyclohexadec-12-en-2-one (habanolide), [2-[1-(3,3-dimethylcyclohexyl)ethoxy]-2-methylpropyl]propanoate (HELVETOLIDE®), (1′R)-3-methyl-5-(2,2,3-trimethylcyclopentan-1-yl)-2-pentanone, (5E)-3-methylcyclopentadec-5-en-1-one (MUSCENONE®), 1,4-dioxacycloheptadecane-5,17-dione (Musk T), 3-methylcyclopentadecan-1-one (L-muscone), 1-(3,5,5,6,8,8-hexamethyl-6,7-dihydronaphthalen-2-yl)ethenone (TONALID®), and combinations thereof.

Non-limiting examples of a piney compound are 1-borneol, 1-bornyl acetate, camphene, camphor gum powder, dihydroterpineol, β-pinene, and combinations thereof.

Examples of a powdery compound include, without limitation, heliotropine and/or whiskey lactone (methyl octalactone).

Non-limiting examples of a spicy compound are acetyl isoeugenol, delta-caryophellene, cardamon oil, cinnamaldehyde, cuminaldehyde, eugenol, isoeugenol, perilla aldehyde, cardamom oil, clove oil, ginger extract, ginger oil, black pepper extract and combinations thereof.

Non-limiting examples of a woody and/or amber compound are amber core, amber extreme, ambretol, 4aR,5R,7aS,9R)-Octahydro-2,2,5,8,8,9a-hexamethyl-4H-4a,9-methanoazuleno[5,6-d]-1,3-dioxole (AMBROCENIDE®), ((3aR,5aS,9aS,9bR)-3a,6,6,9a-Tetramethyldodecahydronaphtho[2,1-b]furan (AMBROXAN), 2-ethyl-4-(2,2,3-trimethyl-3-cyclo-penten-1-yl)-2-buten-1-ol (BACDANOL®), ethoxymethoxycyclododecane (Boisambrene Forte), 1,1,2,3,3-pentamethyl-2,5,6,7-tetrahydroinden-4-one (Cashmeran®), ((2R,5S,7R,8R)-8-methoxy-2,6,6,8-tetramethyltricyclo[5.3.1.01,5]undecane (Cedramber®), cedanol, cedarwood oil, (1S,2R,5S,7R,8R)-2,6,6,8-tetramethyltricyclo[5.3.1.01,5]undecan-8-ol (Cedrol), 2,2,6-trimethyl-alpha-propylcyclohexanepropanol (Dextramber), 3-Methyl-5-(2,2,3-trimethyl-3-cyclopenten-1-yl)-4-penten-2-ol (EBANOL®), (R,E)-2-Methyl-4-(2,2,3-trimethylcyclopent-3-enyl) but-2-en-1-ol (HINDINOL®), hinokitiol, DH-ionone beta, [(1R,2S)-1-methyl-2-[[(1R,3S,5S)-1,2,2-trimethyl-3-bicyclo[3.1.0]hexanyl]methyl]cyclopropyl]methanol (JAVANOL®), 5-butan-2-yl-2-(2,4-dimethyl-1-cyclohex-3-enyl)-5-methyl-1,3-dioxane (karanal), 2,4-Dimethyl-2-(1,1,4,4-tetramethyltetralin-6-yl)-1,3-dioxolane (OKOUMAL®), 1-(1,2,3,4,5,6,7,8-octahydro-2,3,8,8-tatramethyl-2-naphthyl)ethan-1-one (ORBITONE®), patchouly oil, polysantol, rhubofix, sandalwood, and combinations thereof.

The amounts of the fragrance compounds can vary depending on the intended resulting fragrance composition, but can range from about 0.1 parts per thousand to about 800 parts per thousand, or from about 1 part per thousand to about 500 parts per thousand.

Such compositions can contain or consist of at least one ingredient selected from a group consisting of a fragrance carrier and a fragrance base. Such compositions can also consist of at least one fragrance adjuvant.

Fragrance carriers can be a liquid or a solid and typically do not significantly alter the olfactory properties of the fragrance ingredients. Some non-limiting examples of fragrance carriers include an emulsifying system, encapsulating materials, natural or modified starches, polymers, gums, pectins, gelatinous or porous cellular materials, waxes, and solvents which are typically employed in fragrance applications.

Fragrance base refers to any composition comprising at least one fragrance co-ingredient. In general, these co-ingredients belong to chemical classes such as, but not limited to: alcohols, aldehydes, ketones, esters, ethers, acetals, oximes, acetates, nitriles, terpenes, saturated and unsaturated hydrocarbons, and essential oils of natural or synthetic origins.

The fragrance compositions according to the disclosed subject matter can be in the form of a simple mixture of the various co-ingredients and solvents, or also in the form of a biphasic system such as an emulsion or microemulsion. Such systems are well-known to persons skilled in the art.

Nonlimiting examples of such solvents used in perfumery are known in the art and include but are not limited to: dipropyleneglycol, diethyl phthalate, isopropyl myristate, benzyl benzoate, 2-(2ethoxy)-1-ethanol, ethyl citrate, ethanol, water/ethanol mixtures, limonene or other terpenes, isoparaffins such as those known under the trademark Isopar® (ExxonMobil Chemicals, Houston, Tex.), and glycol ethers and glycol ether esters such as those known under the trademark Dowanol© (Dow Chemical Company, Midland, Mich.).

### 5. Use of Fragrance Compositions in Consumer Products

The fragrance compositions of the presently disclosed subject matter as described above can be advantageously used within a wide variety of consumer products. Suitable end products that can include a compound of the presently disclosed subject matter include, but are not limited to: 1) candles, air fresheners, perfumes, and colognes; 2) personal care products such as soaps, deodorants, shampoos, conditioners, shower gels, and shaving lotions; 3) cosmetics such as lotions and ointments; and 4) detergents, fabric care products, and household cleansers/cleaning agents. Depending on the solvents that can be present in some end products, it can be necessary to protect the compounds from premature degradation, for example by encapsulation or with a stabilizer, or other methods well-known to those of ordinary skill in the art.

The above-listing of end products is non-limiting. The compositions of the presently disclosed subject matter can be included in a number of additional products, including for example: 1) fragrance products, eau de perfume, eau de toilet, eau de cologne, and the like; skin-care cosmetics, face washing creams, varnishing creams, cleansing creams, cold creams, massage creams and oils, milky lotions, skin toning lotion, cosmetic solutions, packs, makeup remover, and the like; 2) makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, and the like; 3) hair care cosmetics, pomades, brilliantines, setting lotions, hair sticks, hair solids, hair oils, hair treatments, hair creams, hair tonics, hair liquids, hair sprays, hair restorers, hair dyes, and the like; 4) sunburn cosmetics, suntan products, sunscreen products, and the like; 5) medical cosmetics, antiperspirants, after-shave lotions and gels, permanent wave lotions, medicated soaps, medicated shampoos, medicated skin care products, and the like; 6) hair care products, rinses, shampoo—including-rinses, hair conditioners, hair treatments, hair packs, and the like; 7) as bath soaps, perfumed soaps, transparent soaps, synthetic soaps, and the like; 8) body washing soaps, body soaps, body shampoos, hand soaps, and the like; 9) bathing, bathing agents (e.g., bath salts, bath tablets, bath liquids, and the like), foam baths (bubble bath and the like), bath oils (e.g., bath perfumes, bath capsules, and the like), milk baths, bath gels, bath cubes, and the like; 10) heavy duty detergents for clothes, light duty detergents for clothes, liquid detergents, laundering soaps, compact detergents, powder soaps, and the like; 11) softening finishing agents, softeners, furniture care products, and the like; deodorants, aromatic substances, and the like; 12) insect repellent, insecticides, and the like; 13) oral care products such as tooth pastes, mouth cleaners, mouth wash, troches, chewing gums, and the like; and 14) pharmaceutical products, poultices, external skin care pharmaceuticals such as ointments, internal administration medicines, and the like.

### 6. Flavor Compositions

In certain embodiments, any one of the above-described compounds can be provided in a flavor composition. Certain embodiments of the presently disclosed subject matter provide a method to modify, enhance, or improve the taste properties of a flavor composition by adding to the composition an effective quantity of one or more of the presently disclosed compounds.

For flavor applications, concentrations of the presently disclosed compounds are based on the total weight of the composition into which the flavor compound is incorporated. For flavor applications, typical concentrations of the compounds can range from about 0.0001% to about 20% by weight, or from about 0.01% to about 10% by weight, or from about 0.1% to about 5%, or from about 0.1% to about 5%, based on the total weight of the composition into which the compound is incorporated. Those skilled in the art are able to employ the desired level of said compounds to provide the desired flavor and intensity. Much higher concentrations can be employed when the compounds are used in concentrated flavors and flavor compositions.

As used herein, organoleptic effective quantity will be defined as the amount of said compound in a flavor composition in which the individual component will contribute its characteristic flavor properties. However, the organoleptic effect of the flavor composition will be the sum of the effects of all flavor ingredients present. Therefore, the compounds embodied in the presently disclosed subject matter can be employed to modify the characteristics of the flavor composition via their own organoleptic properties or through additively effecting the contributions of other ingredient(s) present within the said composition. The quantity will vary widely depending on the presence of other ingredients present, their relative amounts, the desired effect, and the nature of the product.

The flavor carrier can be a liquid or a solid, and typically does not significantly alter the olfactory or organoleptic properties of the flavor ingredients, respectively. Some non-limiting examples of flavor carriers include an emulsifying system, encapsulating materials, natural or modified starches, polymers, pectins, proteins, polysaccharides, gums and solvents which are typically employed in flavor applications.

The flavor compositions according to the disclosed subject matter can be in the form of a simple mixture of the various co-ingredients, adjuvants, and solvents, or also in the form of a biphasic system such as an emulsion or microemulsion. Such systems are well-known to persons skilled in the art.

Nonlimiting examples of solvents commonly used in flavors are also known in the art and include, but are not limited to: water, medium-chain triglycerides (MCTs), propylene glycol, triacetin, triethyl citrate, benzyl alcohol, benzyl benzoate, ethanol, vegetable oils, and terpenes.

As used herein, the term “flavor carrier” can also encompass the food, confectionaries, beverage, oral care product, or pharmaceutical to which the flavor compound (i.e., one of the presently disclosed compounds) is added. Examples of such foods, confectionaries, beverages, oral care products, or pharmaceuticals include, but are not limited to carbonated fruit beverages, carbonated cola drinks, wine coolers, cordials, flavored water, powders for drinks (e.g., powdered sports or “hydrating” drinks), hard candy, soft candy, taffy, chocolates, sugarless candies, chewing gum, bubble gum, alcoholic beverages, energy beverages, juices, teas, coffees, breath freshener tablets or drops, film strips for halitosis, gelatin candies, pectin candies, starch candies, lozenges, cough drops, throat lozenges, throat sprays, toothpastes, and mouth rinses.

### 7. Use of Flavor Compositions in Consumer Products

The flavor compositions of the presently disclosed subject matter as described above can be advantageously used within a wide variety of consumer products intended to be eaten, imbibed or otherwise consumed. Suitable end products that can include a compound of the presently disclosed subject matter include but are not limited to: beverages such as juices, sodas, teas, flavored waters, fruit-based “smoothie” drinks, milk-based drinks, and the like; confectionaries such as sweets, hard candy, gums; and pharmaceuticals, oral care products, and the like.

The flavor compositions according to the disclosed subject matter can be in the form of a simple mixture of flavoring ingredients or in an encapsulated form, e.g., a flavoring composition entrapped into a solid matrix that can comprise wall-forming and plasticizing materials such as mono-, di-, or trisaccharides, natural or modified starches, hydrocolloids, cellulose derivatives, polyvinyl acetates, polyvinylalcohols, proteins, or pectins. Examples of particularly useful matrix materials include, for example, sucrose, glucose, lactose, levulose, fructose, maltose, ribose, dextrose, isomalt, sorbitol, mannitol, xylitol, lactitol, maltitol, pentatol, arabinose, pentose, xylose, galactose, maltodextrin, dextrin, chemically modified starch, hydrogenated starch hydrolysate, succinylated or hydrolysed starch, agar, carrageenan, gum arabic, gum accacia, tragacanth, alginates, methyl cellulose, carboxymethyl cellulose, hydroxyethyl cellulose, hydroxypropylmethyl cellulose, derivatives, gelatin, agar, alginate, and mixtures thereof. Encapsulation is well-known to persons skilled in the art, and can be performed, for instance, using techniques such as spray-drying, agglomeration or extrusion, or coating encapsulation, including coacervation and complex coacervation techniques.

In one embodiment, a compound of the presently disclosed subject matter is included/used in chewing and bubble gums and confectionaries (e.g., hard or soft candies or lozenges). Chewing gum compositions typically include one or more gum bases and other standard components such as flavoring agents, softeners, sweeteners, and the like. Flavoring agents for use in chewing gum compositions are well known and include natural flavors such as citrus oils, peppermint oil, spearmint oil, oil of wintergreen, natural menthol, cinnamon, ginger, and the like; and artificial flavors such as menthol, carvone, limonene, cinnamic aldehyde, linalool, geraniol, ethyl butyrate, and the like. As is known in the art, the ingredients used in chewing gum compositions can include sweeteners, both natural and artificial and both sugar and sugarless. Sweeteners are typically present in the chewing gum compositions in amounts of from about 20% to 80% by weight, preferably from about 30% to 60% by weight, based on the total weight of the chewing gum composition. Sugarless sweeteners include, but are not limited sugar alcohols such as Sorbitol, manifold, xylitol, hydrogenated starch hydrolysates, malitol, and the like. High intensity sweeteners such as sucralose, aspartame, neotame, salts of acesulfame, and the like, when employed, are typically present up to about 1.0% by weight.

In an alternative embodiment, a compound of the presently disclosed subject matter is included in an oral personal care product (e.g., a mouthwash or toothpaste). For example, a mouthwash can be prepared by dissolving a flavor composition (e.g., a flavor cocktail) (liquid or powder) that includes a compound of the presently disclosed subject matter in a solvent (e.g., water) that further includes, for example, a flavor such as menthol and a surfactant; and then mixing the resulting solution with, for example, an aqueous erythritol solution.

In one embodiment of the presently disclosed subject matter, a compound of the presently disclosed subject matter is added, directly or indirectly, to a pharmaceutical dosage form (e.g., a tablet, capsule, drop, or lozenge) that contains a therapeutically active agent (e.g., a medicament). For example, one embodiment of the presently disclosed subject matter provides a cough drop or lozenge containing one or more compounds of the present disclosure and, optionally, further containing menthol or other medicaments for the treatment of sore throat, coughing or other upper respiratory ailments.

### EXAMPLES

The present application is further described by means of the examples, presented below, wherein the abbreviations have the usual meaning in the art.

The use of such examples is illustrative only and does not limit the scope and meaning of the disclosed subject matter or of any exemplified term. Likewise, the disclosed subject matter is not limited to any particular preferred embodiments described herein. Indeed, many modifications and variations of the disclosed subject matter are apparent to those skilled in the art upon reading this specification. The disclosed subject matter is therefore to be limited only by the terms of the appended claims along with the full scope of equivalents to which the claims are entitled.

The following Examples are provided as specific embodiments of the present disclosure, wherein the abbreviations have the usual meaning in the art. The temperatures are indicated in degrees centigrade (° C.); RT refers to room temperature (25° C.), the NMR spectral data were recorded in CDCl₃ with a 400 MHz machine for ¹H and ¹³C, the chemical displacements are indicated in ppm with respect to TMS as the standard.

### Example 1: Synthesis of 1-((R)-4-methylcyclohex-1-en-1-yl)ethan-1-ol

1-((R)-4-methylcyclohex-1-en-1-yl)ethan-1-ol was prepared according to the following procedure.

To 1 M LiAlH4 solution in THE (0.6 eq) under nitrogen, a solution of (R)-1-(4-methylcyclohex-1-en-1-yl)ethan-1-one (23.0 g, 1 eq) in MTBE (46 mL) was added drop-wise at RT. The reaction mixture was stirred at RT for 8 h. The reaction was quenched with ice cold water (250 mL) and added MTBE (350 mL). The reaction mixture was stirred for 1 h, filtered through celite and washed with MTBE (2×125 mL). The filtrate was taken into separating funnel and the aqueous layer was extracted with MTBE (2×250 mL). The combined MTBE layers were distilled under vacuum below 45° C. to constant weight to get 22.7 g of the product in 97.2% yield as a mixture of diastereomers (44.89%: 54.39% by GC).

GC/MS (EI): m/z (%) 140 (M⁺, 33), 125 (18), 107 (35), 93 (25), 81 (62), 67 (36), 55 (60), 43 (100). ¹H NMR (CDCl₃): δ 5.61 (1H, v), 4.16 (1H, m), 2.01-2.02 (3H, m), 1.57-1.79 (3H, m), 1.37 (1H, OH), 1.24 (3H, dd) 1.20 (1H, m), 0.9 (3H, dd). ¹³C NMR (CDCl₃): δ 140.5, 121.6, 120.5, 71.8, 31.9, 30.8, 28.5, 23.9, 21.8.

Odor: green, herbaceous, mint, wintergreen.

Flavor (10 ppm ethanol/water): lactonic, creamy, buttery, whiskey, musty, grape.

### Example 2: Synthesis of 1-((R)-4-methylcyclohex-1-en-1-yl)ethyl acetate

1-((R)-4-methylcyclohex-1-en-1-yl)ethyl acetate was prepared according to the following procedure.

A vessel was charged with acetic acid (1.2 eq) in DCM (150 mL) under nitrogen at RT. EDC.HCl (1.2 eq) and DMAP (0.1 eq) were added under nitrogen and stirred at RT for 1 h. 1-((R)-4-methylcyclohex-1-en-1-yl)ethan-1-ol (3.0 g, 1 eq) in DCM (15 mL) was added drop-wise to the reaction mixture. The reaction was stirred at RT for 18 h. The reaction was quenched with ice cold water (30 mL) and added DCM (45 mL). The aqueous layer was extracted with DCM (2×30 mL). The combined DCM layers were washed with water (2×15 mL) followed by sat. NaHCO₃ (2×15 mL). The DCM layer was distilled under vacuum below 45° C. to constant weight to get the crude product which was subjected to column purification to obtain 2.06 g of the product in 52% yield as a mixture of diastereomers (49.98%: 49.35% by GC).

GC/MS (EI): m/z (%) 182 (M⁺, 2), 167 (1), 140 (48), 122 (46), 107 (69), 93 (92), 79 (48), 67 (15), 55 (18), 43 (100). ¹H NMR (CDCl₃): δ 5.63 (1H, v), 5.22 (1H, m), 2.02 (6H, m), 1.56-1.72 (3H, m), 1.27 (3H, d), 1.20 (1H, m), 0.92 (3H, d). ¹³C NMR (CDCl₃): δ 170.5, 136.2, 123.7, 122.6, 74.1, 33.3, 30.6, 28.2, 24.2, 21.6, 18.9.

Odor: Herbaceous, mushroom, cumin.

Flavor (5 ppm ethanol/water): green, waxy, almost rummy, lactone, minty, herbal, celery, root beer, methyl salicylate-like, slightly creamy.

### Example 3: Synthesis of 1-((R)-4-methylcyclohex-1-en-1-yl)ethyl propionate

1-((R)-4-methylcyclohex-1-en-1-yl)ethyl propionate acetate was prepared according to the following procedure.

The same procedure as described in Example 2 was followed, using propionic acid instead of acetic acid. After silica gel column chromatography, 0.96 g of the product was obtained in 68.5% yield as a mixture of diastereomers (46.25%: 51.66% by GC).

GC/MS (EI): m/z (%) 196 (M⁺, 2), 179(1), 167 (2), 140 (53), 122 (47), 107 (70), 93 (100), 79 (58), 57 (71), 41 (44). ¹H NMR (CDCl₃): δ 5.58 (1H, v), 5.19 (1H, m), 2.25 (2H, q), 1.96 (3H, m), 1.49-1.78 (4H, m), 1.22 (3H, d), 1.10 (3H, m), 0.87 (3H, d). ¹³C NMR (CDCl₃): δ 173.7, 135.9, 123.6, 122.4, 73.3, 33.4, 30.7, 27.9, 24.0, 21.5, 18.7, 9.2.

Odor: green, mushroom, fruity, green pear, herbaceous.

Flavor (2 ppm ethanol/water): soapy, cilantro, fatty, creamy mouthfeel, mushroom, slightly fruity, minty, cooling, green, aldehydic, herbal, earthy, like asafetida.

### Example 4: Synthesis of 1-((R)-4-methylcyclohex-1-en-1-yl)ethyl cyclopropanecarboxylate

1-((R)-4-methylcyclohex-1-en-1-yl)ethyl cyclopropanecarboxylate acetate was prepared according to the following procedure.

The same procedure as described Example 2 was followed using cyclopropane carboxylic acid instead of acetic acid. After silica gel column chromatography, 2.09 g of the product was obtained in 47% yield as a mixture of diastereomers (43.81%: 51.68% by GC).

GC/MS (EI): m/z (%) 207 (M⁺-1, 4), 193 (1), 139 (4), 122 (29), 107 (36), 93 (55), 69 (100), 55 (13), 41 (66). ¹H NMR (CDCl₃): δ 5.57 (1H, v), 5.18 (1H, m), 1.96 (3H, m), 1.53 (1H, m), 1.52 (3H, m), 1.22 (3H, d), 1.14 (1H, m), 0.88 (5H, m), 0.76 (2H, m). ¹³C NMR (CDCl₃): δ 174.2, 137.5, 123.5, 122.4, 73.9, 33.5, 30.6, 28.2, 24.2, 21.6, 19.0, 13.1, 8.27.

Odor: bleu cheese, pear, leafy.

### Example 5: Synthesis of 1-((R)-4-methylcyclohex-1-en-1-yl)ethyl benzoate

1-((R)-4-methylcyclohex-1-en-1-yl)ethyl benzoate was prepared according to the following procedure.

Same procedure as described in Example 2 was followed using benzoic acid instead of acetic acid. After silica gel column chromatography, 0.88 g of the product was obtained in 50% yield.

GC/MS (EI): m/z (%) 244 (M⁺, 2), 207 (2), 139 (7), 122 (26), 105 (100), 93 (34), 77 (44), 65 (4), 51 (16), 39 (10). ¹H NMR (CDCl₃): δ 8.02 (2H, ar), 7.50 (1H, ar), (7.41 (2H, ar), 5.74 (1H, v), 5.49 (1H, m), 1.96 (3H, m), 1.75 (1H, m), 1.62 (2H, m), 1.41 (3H, d), 1.27, (1H, m), 0.93 (3H, d). ¹³C NMR (CDCl₃): δ 168.5, 136.1, 132.7, 130.9, 129.5, 123.8, 122.7, 74.3, 33.3, 30.6, 28.2, 24.1, 21.6, 19.2.

Odor: mothball, phenolic.

Flavor (20 ppm in ethanol/water): creamy, waxy, fatty and buttery linger, fruity, grape, salicylate, phenolic, medicinal, sweet, brown, molasses-like, geranial, mushroom.

### Example 6: Synthesis of (4R)-1-(1-methoxyethyl)-4-methylcyclohex-1-ene

(4R)-1-(1-methoxyethyl)-4-methylcyclohex-1-ene was prepared according to the following procedure.

To 60% NaH dispersion in mineral oil (1.5 eq) in THE (30 mL) under nitrogen, 1-((R)-4-methylcyclohex-1-en-1-yl)ethan-1-ol (2.0 g, 1 eq) dissolved in THF (10 mL) was added at 0-5° C. and stirred for 1 h. Me (2.0 eq) was added to the reaction mixture and allowed to warm to RT. The reaction was maintained at RT for 18 h. The reaction was cooled to 0-5° C. and quenched with water (20 mL) and extracted with MTBE (2×20 mL). The combined MTBE layers were distilled under vacuum to give the crude product. The crude material was purified by silica gel column chromatography to give a mixture of diastereomers (42.43%: 56.44% by GC) of the methyl ether (1.25 g, 57% yield). GC/MS (EI): m/z (%) 154 (M⁺, 52), 139 (77), 122 (11), 107 (100), 93 (40), 79 (70), 59 (81, 41 (56). ¹H NMR (CDCl₃): δ 5.62 (1H, vinyl, m), 3.69 (1H, m), 3.22 (3H, s), 1.95-2.19 (3H, m), 1.62-1.77 (3H, m), 1.32 (4H, m), 1.02 (3H, d). ¹³C NMR (CDCl₃): δ 137.7, 124.2, 123.5, 81.3, 55.6, 33.4, 30.6, 28.4, 22.3, 19.8.

Odor: horseradish root, wasabi.

### Example 7: Synthesis of (R)-1-(4-methylcyclohex-1-en-1-yl)ethan-1-one oxime

(R)-1-(4-methylcyclohex-1-en-1-yl)ethan-1-one oxime was prepared according to the following procedure.

A vessel was charged with (R)-1-(4-methylcyclohex-1-en-1-yl)ethan-1-one (1.0 g, 1 eq), NH₂OH.HCl (1.7 eq) in EtOH (20 mL) and pyridine (2 eq) under nitrogen at RT. The reaction was heated to reflux and maintained at reflux for 18 h. The reaction mixture was subjected to distillation to remove solvent and the crude was partitioned between water (10 mL) and EtOAc (10 mL). The aqueous layer was extracted with EtOAc (2×10 mL). The combined EtOAc layers were distilled under vacuum below 45° C. to constant weight to get crude product. Water (50 mL) was added to the crude and stirred at RT for 1 h. The solids were washed with water (2×0 mL) and dried under vacuum below 45° C. to constant weight to get 0.86 g of the product in 78.2% yield.

GC/MS (EI): m/z (%) 153 (M⁺, 47), 136 (18), 121 (29), 110 (42), 94 (43), 79 (62), 67 (37), 53 (51), 42 (100). ¹H NMR (CDCl₃): δ 9.469 (1H, OH), 6.13 (1H, v), 2.40 (1H, m), 2.21 (2H, m), 2.20 (3H, dd), 1.75 (2H, m), 1.63 (1H, m), 1.20 (1H, m), 0.93 (3H, dd). ¹³C NMR (CDCl₃): δ 156.7, 134.0, 129.6, 34.4, 30.5, 28.0, 24.2, 21.5, 9.7.

Odor: weak.

### Example 8: Synthesis of (R)-2-methyl-2-(4-methylcyclohex-1-en-1-yl)-1,3-dioxolane

(R)-2-methyl-2-(4-methylcyclohex-1-en-1-yl)-1,3-dioxolane was prepared according to the following procedure.

(R)-1-(4-methylcyclohex-1-en-1-yl)ethan-1-one (2.0 g, 1 eq), ethylene glycol (12 eq), toluene (40 mL) and p-TsOH (0.04 eq) were combined under nitrogen at RT. The reaction was heated to reflux for 18 h. The solvent was removed and the reaction then quenched with water (20 mL) and extracted with MTBE (2×40 mL). Concentration of the solvent gave the crude product, which was purified by silica gel column chromatography to give the product (1.6 g, 61% yield) as a mixture of diastereomers (83.95%:14.74% by GC).

GC/MS (EI): m/z (%) 181 (M⁺-1 1), 167 (100), 139 (2), 125 (13), 109 (2), 87 (68), 67 (7), 43 (49). ¹H NMR (CDCl₃): δ 5.87 (1H, vinyl, m), 3.95-3.99 (3H, m), 3.82-3.89 (2H, m), 2.11-2.17 (3H, m), 2.04-2.07 (2H, m), 1.91 (1H, m), 1.70-1.78 (1H, m), 1.37-1.41 (2H, m), 0.99 (3H, d). ¹³C NMR (CDCl₃): δ 137.1, 126.2, 122.1, 109.3, 64.3, 64.1, 33.3, 30.9, 28.3, 23.7, 21.6.

Odor: green, floral, earthy, bitter green, chrysanthemum, slightly mossy.

### Example 9: Use of 1-((R)-4-methylcyclohex-1-en-1-yl)ethan-1-ol in a Fragrance Formulation for a Fresh Scent Liquid Laundry Detergent

The present Example provides a fragrance formulation for a fresh scent liquid laundry detergent. Table 1 provides a summary of the category of components of the fragrance formula.

TABLE 1

| CATEGORY | AMOUNT (PPM) |
|---|---|
| Aldehydic | 4 |
| Citrus | 272 |
| Floral | 280 |
| Fruity | 166 |
| Example 1 | 2 |
| Green | 1 |
| Musk | 74 |
| Solvent | 107 |
| Woody/amber | 94 |
| Total | 1000 |

### Example 10: Use of 1-((R)-4-methylcyclohex-1-en-1-yl)ethyl Propionate in a Formulation for a Hazelnut Flavor

The present Example provides comparison of flavor compositions with and without 1-((R)-4-methylcyclohex-1-en-1-yl)ethyl propionate as outlined in Table 2.

TABLE 2

| | A | B |
|---|---|---|
| Compound Name | (%) | (%) |
| hexyl aldehyde | 0.020 | 0.020 |
| methyl heptenone | 0.020 | 0.020 |
| 5-methyl 2-hepten-4-one | 0.070 | 0.070 |
| 2-methyl butyric acid | 0.005 | 0.005 |
| Vanillin | 0.020 | 0.020 |
| Compound of Example 3 | — | 0.010 |
| Ethanol | 99.865 | 99.855 |
| | 100.000 | 100.000 |

Composition A was developed by Kieft et al. and published in article “1. Sensomics Analysis of Key Hazelnut Odorants (Corylus avellana L. ‘Tonda Gentile’) Using Comprehensive Two-Dimensional Gas Chromatography in Combination with Time-of-Flight Mass Spectrometry (GC×GC-TOF-MS)”, J. Agric. Food Chem. 2013 61 (22), 5226-5235. Composition B contained the same components as Composition A, but additionally included 1-((R)-4-methylcyclohex-1-en-1-yl)ethyl propionate. Each of the compositions was dosed in water at 0.10% and evaluated by two Flavorists. It was found that addition of 1-((R)-4-methylcyclohex-1-en-1-yl)ethyl propionate boosted the hazelnut character to the front, and added creaminess.

### Example 11: Use of 1-((R)-4-methylcyclohex-1-en-1-yl)ethan-1-ol in a Formulation for a Butter Flavor

The present Example provides comparison of flavor compositions with and without 1-((R)-4-methylcyclohex-1-en-1-yl)ethan-1-ol as outlined in Table 3.

TABLE 3

| | C | D |
|---|---|---|
| Compound Name | (%) | (%) |
| Diacetyl | 0.030 | 0.030 |
| Delta decalactone | 0.250 | 0.250 |
| Butyric acid | 0.220 | 0.220 |
| Compound of Example 1 | — | 0.100 |
| Ethanol | 99.500 | 99.400 |
| | 100.000 | 100.000 |

Composition C was adapted from Flavourings Production, Composition, Applications, Regulations by Erich Ziegler and Herta Ziegler. Composition D included the same components as Composition C and additionally 1-((R)-4-methylcyclohex-1-en-1-yl)ethan-1-ol. This flavor can be evaluated in water at 0.10%.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method, and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entireties for all purposes.

1. A compound represented by Formula I: 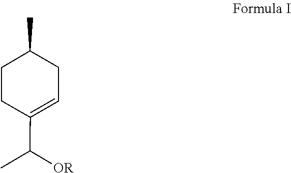
where R is H, C₁-C₈ alkyl, C₁-C₈ alkenyl, C(O)R₁, or C(O)OR₁; and
where R₁ is selected from the group consisting of H, C₁-C₈ alkyl, C₁-C₈ branched alkyl, C₁-C₈ alkenyl, C₁-C₈ branched alkenyl, C₁-C₈ cycloalkyl, aryl, and a substituted aryl.

2. A compound represented by Formula II: 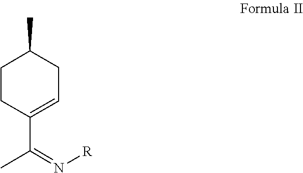
where R is H or OR₁, and
where R₁ is selected from the group consisting of C₁-C₈ alkyl, C₁-C₈ branched alkyl, C₁-C₈ alkenyl, C₁-C₈ branched alkenyl, and C(O)R₂; and R₂ is selected from the group consisting of H, C₁-C₅ alkyl, C₁-C₅ branched alkyl, C₁-C₅ alkenyl, and C₁-C₅ branched alkenyl.

3. (canceled)

4. A compound represented by Formula IIIa: 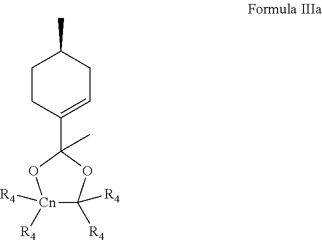
wherein n is 1 or 2; and
wherein each R₄ is independently H, C₁-C₈ alkyl, C₁-C₈ branched alkyl, C₁-C₈ alkenyl, or C₁-C₈ branched alkenyl.

5. A fragrance composition comprising at least one compound of claim 1.

6. The fragrance composition of claim 5, wherein the concentration of the at least one compound is from about 0.001% to about 20% by weight of the fragrance composition.

7. The fragrance composition of claim 5, further comprising one or more compounds selected from the group consisting of one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

8. A flavor composition comprising at least one compound of claim 1.

9. The flavor composition of claim 8, wherein the concentration of the at least one compound is from about 0.0001% to about 20% by weight of the flavor composition.

10. The flavor composition of claim 8, further comprising a flavor carrier.
